SEQUENCE LISTING (4 sequences)

SEQ ID NO: 1 - DNA, 33 bp, Artificial
  Oligonucleotide
gccgaattcatgccatcggcagatcagatcctg

SEQ ID NO: 2 - DNA, 34 bp, Artificial
  Oligonucleotide
taatctagatcaattcaggcccagaagtcgcttg

SEQ ID NO: 3 - DNA, 1806 bp, Drosophila melanogaster
atgccatcggcagatcagatcctgtttgtaaatgtcaccacaacggtggcggcggcggctctaaccgctgcggccgccgtcagcaccacaaagtccggaagcggcaacgccgcacggggctacacggattcggatgacgatgcgggcatgggaacggaggcggtggctaacatatccggctcgctggtggagggcctgaccaccgttaccgcggcattgagtacggctcaggcggacaaggactcagcgggagaatgcgaaggagctgtggaggagctgcatgccagcatcctgggcctccagctggctgtgccggagtgggaggcccttctcaccgccctggttctctcggtcattatcgtgctgaccatcatcgggaacatcctggtgattctgagtgtgttcacctacaagccgctgcgcatcgtccagaacttcttcatagtttcgctggcggtggccgatctcacggtggcccttctggtgctgcccttcaacgtggcttactcgatcctggggcgctgggagttcggcatccacctgtgcaagctgtggctcacctgcgacgtgctgtgctgcactagctccatcctgaacctgtgtgccatagccctcgaccggtactgggccattacggaccccatcaactatgcccagaagaggaccgttggtcgcgtcctgctcctcatctccggggtgtggctactttcgctgctgataagtagtccgccgttgatcggctggaacgactggccggacgagttcacaagcgccacgccctgcgagctgacctcgcagcgaggctacgtgatctactcctcgctgggctccttctttattccgctggccatcatgacgatcgtctacatcgagatcttcgtggccacgcggcgccgcctaagggagcgagccagggccaacaagcttaacacgatcgctctgaagtccactgagctcgagccgatggcaaactcctcgcccgtcgccgcctccaactccggctccaagtcgcgtctcctagccagctggctttgctgcggccgggatcgggcccagttcgccacgcctatgatccagaacgaccaggagagcatcagcagtgaaacccaccagccgcaggattcctccaaagcgggtccccatggcaacagcgatccccaacagcagcacgtggtcgtgctggtcaagaagtcgcgtcgcgccaagaccaaggactccattaagcacggcaagacccgtggtggccgcaagtcgcagtcctcgtccacatgcgagccccacggcgagcaacagctcttacccgccggcggggatggcggtagctgccagcccggcggaggccactctggaggcggaaagtcggacgccgagatcagcacggagagcgggagcgatcccaaaggttgcatacaggtctgcgtgactcaggcggacgagcaaacgtccctaaagctgaccccgccgcaatcctcgacgggagtcgctgccgtttctgtcactccgttgcagaagaagactagtggggttaaccagttcattgaggagaaacagaagatctcgctttccaaggagcggcgagcggctcgcaccctgggcatcatcatgggcgtgttcgtcatctgctggctgcccttcttcctcatgtacgtcattctgcccttctgccagacctgctgccccacgaacaagttcaagaacttcatcacctggctgggctacatcaactcgggcctgaatccggtcatctacaccatcttcaacctggactaccgccgggccttcaagcgacttctgggcctgaattga

SEQ ID NO: 4 - protein, 601 aa, Drosophila melanogaster
MPSADQILFVNVTTTVAAAALTAAAAVSTTKSGSGNAARGYTDSDDDAGMGTEAVANISGSLVEGLTTVTAALSTAQADKDSAGECEGAVEELHASILGLQLAVPEWEALLTALVLSVIIVLTIIGNILVILSVFTYKPLRIVQNFFIVSLAVADLTVALLVLPFNVAYSILGRWEFGIHLCKLWLTCDVLCCTSSILNLCAIALDRYWAITDPINYAQKRTVGRVLLLISGVWLLSLLISSPPLIGWNDWPDEFTSATPCELTSQRGYVIYSSLGSFFIPLAIMTIVYIEIFVATRRRLRERARANKLNTIALKSTELEPMANSSPVAASNSGSKSRLLASWLCCGRDRAQFATPMIQNDQESISSETHQPQDSSKAGPHGNSDPQQQHVVVLVKKSRRAKTKDSIKHGKTRGGRKSQSSSTCEPHGEQQLLPAGGDGGSCQPGGGHSGGGKSDAEISTESGSDPKGCIQVCVTQADEQTSLKLTPPQSSTGVAAVSVTPLQKKTSGVNQFIEEKQKISLSKERRAARTLGIIMGVFVICWLPFFLMYVILPFCQTCCPTNKFKNFITWLGYINSGLNPVIYTIFNLDYRRAFKRLLGLN

(19) United States
(12) Patent Application Publication
(10) Pub. No.: US 2011/0003317 A1
(43) Pub. Date: Jan. 6, 2011

(54) METHODS OF SCREENING COMPOUNDS FOR INSECT-CONTROL ACTIVITY INVOLVING THE TYRAMINE RECEPTOR

(75) Inventor: Essam Enan, Nashville, TN (US)

Correspondence Address: DAVIS WRIGHT TREMAINE LLP - San Francisco, 505 MONTGOMERY STREET, SUITE 800, SAN FRANCISCO, CA, 94111, US

(73) Assignee: TYRATECH, INC., Melbourne, FL (US)

(21) Appl. No.: 12/822,139

(22) Filed: Jun. 23, 2010

Related U.S. Application Data
(63) Continuation of application No. 11/365,426, filed on Mar. 1, 2006, now abandoned.
Application No. 11/365,426 is a continuation-in-part of application No. 11/086,615, filed on Mar. 21, 2005, now Pat. No. 7,622,269.
Application No. 11/365,426 is a continuation-in-part of application No. 10/832,022, filed on Apr. 26, 2004, now Pat. No. 7,541,155.
(60) Provisional application No. 60/657,515, filed on Mar. 1, 2005.
(60) Provisional application No. 60/554,968, filed on Mar. 19, 2004.
(60) Provisional application No. 60/532,503, filed on Dec. 24, 2003.
(60) Provisional application No. 60/465,320, filed on Apr. 24, 2003.

(51) Int. Cl. G01N 33/567

(52) U.S. Cl. 435/7.21

(57) ABSTRACT

An exemplary method of screening compositions for insect control activity includes, providing an insect cell expressing a receptor of the insect olfactory cascade or fragment thereof, contacting a test composition to the insect cell, measuring at least one parameter selected from olfactory cascade receptor binding affinity, intracellular cAMP levels, and intracellular Ca²⁺ levels, and selecting a compound capable of altering at least one of parameter selected from increased olfactory cascade receptor binding affinity, altered intracellular cAMP levels, and altered intracellular Ca²⁺ levels. An exemplary isolated eukaryotic cell is transformed with a nucleic acid encoding an insect olfactory cascade receptor protein or fragment thereof. An exemplary method for controlling an insect includes, contacting a composition including a compound having a binding affinity for an olfactory cascade receptor of an insect.

### CROSS-REFERENCE TO RELATED APPLICATIONS

This application is a continuation of U.S. patent application Ser. No. 11/365,426, filed on Mar. 1, 2006. U.S. patent application Ser. No. 11/365,426 claims priority from U.S. Provisional Application No. 60/657,515 filed Mar. 1, 2005, and is a continuation-in-part of commonly assigned U.S. patent application Ser. No. 10/832,022 filed Apr. 26, 2004, now U.S. Pat. No. 7,541,155, which claimed priority from U.S. Provisional Application No. 60/465,320 filed Apr. 24, 2003 and U.S. Provisional Application No. 60/532,503 filed Dec. 24, 2003; and is also a continuation-in-part of commonly assigned and U.S. patent application Ser. No. 11/086,615 filed Mar. 21, 2005, now U.S. Pat. No. 7,622,269, which was a continuation-in-part of commonly assigned U.S. patent application Ser. No. 10/832,022, filed on Apr. 26, 2004, now U.S. Pat. No. 7,541,155, and which further claimed priority from U.S. Provisional Application No. 60/554,968 filed Mar. 19, 2004. The entire disclosure of each of the foregoing applications is incorporated herein by reference in its entirety.

### FIELD OF THE INVENTION

The present invention relates to compositions, methods, cell lines and reports related to controlling insects.

### BACKGROUND OF THE INVENTION

Animals have chemosensory and mechanosensory systems that recognize a large array of environmental stimuli, generating behavioral responses. Behavioral studies have been conducted to understand the genetics of these systems. The olfactory system plays a role in the survival and maintenance of species, including insects.

Drosophila is one of the models for studying the sensory system, as it is amenable to mutant analysis using molecular techniques, behavioral analysis, and electrophysiological analysis, and because its olfactory system is comparable to the mammalian counterpart.

Various chemicals and mixtures have been studied for pesticidal activity for many years with a goal of obtaining a product which is selective for invertebrates such as insects and has little or no toxicity to vertebrates such as mammals, fish, fowl and other species and does not otherwise persist in and damage the environment.

Most of the previously known and commercialized products having sufficient pesticidal activity to be useful also have toxic or deleterious effects on mammals, fish, fowl or other species which are not the target of the product. For example, organophosphorus compounds and carbamates inhibit the activity of acetylcholinesterase in insects as well as in all classes of animals. Chlordimeform and related formamidines are known to act on octopamine receptors of insects but have been removed from the market because of cardiotoxic potential in vertebrates and carcinogenicity in animals and a varied effect on different insects. Other compounds, which may be less toxic to mammals and other non-target species, are sometimes difficult to identify as efficacious for such use.

### SUMMARY OF THE INVENTION

Octopamine (OA) and tyramine (TA) are neuroactive ligands that are ubiquitous and occur in large amounts in invertebrates. See e.g., Evans, et al., (1980) Nature 287, 60-62; Roeder (1992) Life Sci. 50, 21-28. Both OA and TA are members of the subfamily of biogenic amines, and their receptors belong to the superfamily of G-protein coupled receptors (GPCRs). See e.g., Kravitz, et al., (1976) Neurosci. Symp. 1, 67-81; Robertson, et al. (1976) Int. Rev. Neurobiol. 19, 173-224; Orchard (1982) Can. J. Zool. 60, 659-669; Vernier, et al., (1995) Trends Pharmacol. Sci. 16, 375-385; Blenau, et al., (2001) Arch. Insect Biochem. Physiol. 48, 13-38. The activation of GPCRs leads to change of intracellular concentrations of second messengers [cAMP]ᵢ and/or [Ca²⁺]ᵢ. Since these are the most commonly found cellular responses to treatment with biogenic amines, they are used to functionally classify receptor subtypes. See Blenau, et al., (2001), supra.

In insects, OA is synthesized from TA by hydroxylation on the β-carbon side chain and TA is formed by decarboxylation of tyrosine, therefore, TA is considered the direct precursor of OA. See e.g., Roeder (1994) Comp. Biochem. Physiol. 107C, 1-12; Vanden Broeck, et al., (1995) J. Neurochem. 64, 2387-2395. Due to the importance of both ligands in insect biology, cloning and pharmacological analysis of octopamine receptors and tyramine receptors from particular insect species is of interest to the field of insect control. See e.g., Bischof, et al., (2004) Insect Biochem. Mol. Biol. 34, 511-521; Enan (2005) Insect Biochem. Mol. Biol., accepted for publication. The role of OA as a neurotransmitter, a neurohormone, and as a neuromodulator, has been established. See Roeder (1999) Prog. Neurobiol. 59, 533-561 review; and Blenau, et al., (2001) Arch. Insect Biochem. Physiol. 48, 13-38 review. Additionally, it has been suggested that TA plays a possible role as a neurotransmitter and neuromodulator in locust oviducts as well as in different tissues of other insect species. See Downer, et al., (1994) Biogenic amines in insects, in Insect Neurochemistry and Neurophysiology 1993 (Borkovec A. B. and Loeb M. J., eds), pp. 23-38. CRC Press, Boca Raton, Fla.; Downer, et al., (1993) Neurochem. Res. 18, 1245-1248; Roeder (1994) Comp. Biochem. Physiol. 107C, 1-12; Vanden Broeck, et al., (1995) J. Neurochem. 64, 2387-2395; Kutsukake, et al., (2000) Gene 245, 31-42; Ohta, et al., (2003) Insect Mol. Biol. 12(3), 217-223; Donini, et al., (2004) J. Insect Physiol. 50, 351-361. TA has also been shown to have a role in insect olfaction. See Kutsukake, et al., (2000), supra.

Pharmacologically, the tyramine receptors are distinct from the known octopamine receptors, in particular for their binding affinity to the antagonist yohimbine as opposed to other biogenic amine receptor antagonists. See e.g., Arakawa, et al., (1990) Neuron 2, 343-354; Saudou, et al., (1990) EMBO J. 9, 3611-3617; Vanden Broeck, et al., (1995), supra; Ohta, et al., (2003), supra.

Plant essential oils are naturally occurring substances, which are often responsible for a plant's distinctive scent or taste. There are about 17,500 aromatic compounds that occur in higher plants. See Bruneton (1999) Pharmacognosy, phytochemistry, Medicinal Plants: Essential oils 2^{nd} ed. Lavoisier Publishing, New York, pp. 461-780. Essential oils accumulate in vegetative organs such as flowers (bergamot tree, tuberose), leaves (citronella, eucalyptus), barks (cinnamon), woods (rosewood, sandalwood), roots (vetiver), rhizomes (turmeric, ginger), fruits (allspice, anise, star anise) and seeds (nutmeg). In most cases, the biological function of the essential oils remain obscure. It is conceivable, however, that they do have an ecological role. For example, some plant essential oil monoterpenoids are found to possess insecticidal activity as well as attractant, repellent, feeding deterrents, and ovipositional stimulant activities against various insect species. Sangwan, et al., (1990) Pestic. Sci. 28, 331-335; Karr, et al, (1992) J. Econ. Entomol. 85, 424-429; Rice and Coats (1994) J. Econ. Entomol. 87, 1172-1179; Rice and Coats (1994) Structural requirements for monoterpenoid activity against insects. pp. 92-108. In Hedin, P. A. [ed], Bioregulators for crop protection and pest control. Amer. Chem. Soc., Washington, D.C.; Coats, et al., (1991) Toxicity and neurotoxic effects of monoterpenoids in insects and earthworms, pp. 305-316. In P. A. Hedin [ed], Naturally occurring pest bioregulators. Amer. Chem. Soc., Washington, D.C.; Lee, et al., (1997) Ecotoxicol. 90 (4), 883-892; Ngoh, et al., (1998) Pestic. Sci. 54, 261-268; Hori (1999) Appl. Entomol. Zool. 34 (3), 351-358; Landolt et al., (1999) Environ. Entomol. 28(6), 954-960; Sawamura, et al, (1999) J. Agric. Food Chem. 47, 4868-4872; Enan (2001) Comp. Biochem. Physiol. C Toxicol. Pharmacol. 130, 325-337; Enan (1998) Insecticidal action of terpenes and phenols to the cockroaches: effect on octopamine receptors. International Symposium on Crop Protection, Gent, Belgium, May., Abou El Ele and Enan (2002) Insecticidal activity of some essential oils: cAMP mediates effect. Bulletin of High Institute of Public Health, University of Alexandria, Alexandria, Egypt. 31(1), 15-30.

Monoterpenoids of plant essential oils are neurotoxicants against different insect species. See Coats, et al., (1991) Toxicity and neurotoxic effects of monoterpenoids in insects and earthworms, pp. 305-316. In P. A. Hedin 8 [ed], Naturally occurring pest bioregulators. Amer. Chem. Soc., Washington, D.C.; and Enan (1998), supra. They have been shown to inhibit both the GABA receptor in marine algi (Coats (1990) Environ. Health Prespect. 87, 255-262), and inhibit acetylcholinesterase (AChE) isolated from different insect species (Grundy and Still (1985) Pestic. Biochem. Physiol. 3, 383-388; Ryan and Byrne (1988) J. Chem. Eco. 14, 965-1975), and bovine erythrocytes (Miyazawa, et al., (1997) J. Agric. Food Chem. 45, 677-679). However, no correlation was found between in vivo inhibition of AChE and the toxicity of monoterpenoids.

The biological and molecular role of biogenic amine receptors with respect to the mode of action and toxicity of plant essential oils is an aspect of the present invention and is described herein in greater detail.

Aspects of the present invention are described and tested as summarized in this paragraph. Because TA is the immediate precursor of OA, and due to the significance of the tyramine receptor in the biology of different insect species, the molecular role of this receptor in the insecticidal activity of certain plant essential oils is analyzed. In this regard, cDNA coding the tyramine receptor (TyrR) from Drosophila melanogaster is characterized. A stably transfected clonal cell line (pAC-TyrR) is developed and used in the assessment of the structure activity relationships of certain plant essential oils that are structurally related. Whether the tyramine receptor mediated the insecticidal activity of tested plant essential oils is assessed by determining their toxicity against the wild type and tyramine receptor mutant (TyrR^{neo30}) Drosophila melanogaster strains. Data indicate a correlation between cellular changes and insecticidal activity of tested plant essential oils. An optimal chemical structure appears to contribute to their toxicity as judged by the differences in their LD₅₀ values. In addition, the data demonstrate that the insecticidal activity of two isomeric phenolic derivatives, thymol and carvacrol, of monoterpenoid p-cymene is mediated through the tyramine receptor.

In an embodiment of the present invention, a method of screening compositions for insect control activity is provided, by providing an insect cell expressing a receptor of the insect olfactory cascade or fragment thereof, contacting a test composition to the insect cell, measuring at least one parameter selected from: olfactory cascade receptor binding affinity, intracellular cAMP levels, and intracellular Ca²⁺ levels, and selecting a compound capable of altering at least one parameter selected from increased olfactory cascade receptor binding affinity, altered intracellular cAMP levels, and altered intracellular Ca²⁺ levels.

In another embodiment of the present invention, an isolated eukaryotic cell transformed with a nucleic acid encoding an insect olfactory cascade receptor protein or fragment thereof is provided.

In yet another embodiment of the present invention, a method for controlling an insect is provided, by contacting with a composition having a compound with a binding affinity for an olfactory receptor of the insect.

In a further embodiment of the present invention, a method of screening compositions for insect control activity is provided, by providing an insect cell expressing a tyramine receptor or fragment thereof, contacting a test composition to the insect cell, measuring at least one parameter selected from: tyramine receptor binding affinity, intracellular cAMP levels, and intracellular Ca²⁺ levels, and selecting a compound capable of altering at least one parameter selected from: increased tyramine receptor binding affinity, altered intracellular cAMP levels, and altered intracellular Ca²⁺ levels. The contacting of the composition to the insect cell can increase tyramine receptor binding affinity. The contacting of the composition to the insect cell can alter the level of intracellular cAMP. The contacting of the composition to the insect cell can increase the level of intracellular Ca²⁺. The insect cell can be a Drosophila Schneider 2 (S2) cell. The insect cell can have been transformed with a nucleic acid encoding a tyramine receptor or fragment thereof.

In yet another embodiment of the present invention, an isolated eukaryotic cell transformed with a nucleic acid encoding a tyrR receptor protein or fragment thereof is provided. The eukaryotic cell can be an insect cell. The insect cell can be, for example, a Drosophila Schneider 2 (S2) cell.

In yet another embodiment of the present invention, a method for controlling an insect is provided, by contacting a composition including a compound having a binding affinity for the tyramine receptor to an insect. The controlling an insect can occur, for example, by at least one of the following: repellant effect, pesticidal effect, and toxicity. The composition can repel the insect. The composition can be toxic to the insect. The step of contacting the composition to the insect can result in insect mortality. In some embodiments, a step of contacting the composition to a mutant insect strain having a nonfunctional tyrR receptor does not result in insect mortality. The compound can be derived from a plant. The compound can be a plant essential oil. The compound can be selected from, for example, tyramine, p-cymene, thymol, L-carvone, a-terpineol, carvacrol, linalool, arbanol, thyme oil, lilac flower oil, and black seed oil. The step of contacting the compound to the insect can alter the level of intracellular cAMP. The step of contacting the compound to the insect can alter the level of intracellular Ca²⁺. The insect can be contacted, for example, with at least one additional insect control agent.

### DESCRIPTION OF THE DRAWINGS

FIG. 1 depicts chemical structures for tyramine and other monoterpenoid plant essential oils;

FIG. 2 is a bar graph showing the specific binding of ³H-tyramine to TyrR; wherein membrane fractions prepared from S2 cells transfected with either the plasmid (pAC, first bar) lacking the insert (TyrR) or pAC-TyrR (second bar) are analyzed for binding to 4 nM ³H-tyramine and specific binding is calculated by determining nonspecific binding with 10 μM TA and subtracting nonspecific binding from total binding;

FIG. 3 depicts a saturation binding curve of ³H-tyramine to TyrR; wherein membrane fractions prepared from S2 cells expressing pAC-TyrR are analyzed for binding to ³H-tyramine from a range of 0.1-30 nM and specific binding is calculated by determining nonspecific binding with 10 μM TA and subtracting nonspecific binding from total binding;

FIG. 4 depicts an inhibition binding curve of ³H-tyramine to membranes prepared from S2 cells expressing pAC-TyrR; wherein membranes are incubated with 4 nM ³H-tyramine in the presence of unlabeled ligands at a range of concentrations;

FIG. 5 is a bar graph depicting the effect of TA on cAMP levels in S2 cells expressing TyrR; wherein S2 cells stably expressing TyrR are treated with 300 μM IBMX and the effect of TA (10 μM) on basal or forskolin (FK)-increased cAMP levels is measured;

FIG. 6 is a graph depicting the effect of TA on intracellular calcium [Ca²⁺]ᵢ levels in S2 cells either transfected with the plasmid (pAC) lacking the insert (TyrR) or stably expressing pAC-TyrR; wherein S2 Cells are incubated for 60 s before the addition of 1 μM TA, the arrow indicates addition of TA and the fluorescence ratio determined from excitation with 340 nm and 380 nm is plotted to indicate transient increase in [Ca²⁺]i levels;

FIG. 7 is a bar graph showing that the chelation of intracellular calcium does not inhibit the TA-mediated decrease in cAMP; wherein S2 cells expressing TyrR are incubated with 20 μM BAPTA/AM to chelate intracellular calcium for 10 min prior to the addition of TA (0.1 and 1 μM), cAMP levels are determined from cells treated with TA in the presence and absence of BAPTA/AM, and cAMP levels are plotted to indicate changes in cAMP levels;

FIG. 8 is a bar graph depicting the inhibitory effect of tested plant essential oils on the binding of ³H-tyramine to membranes prepared from S2 cells expressing pAC-TyrR; wherein membranes are incubated with 4 nM ³H-tyramine in the presence of tested plant essential oils, specific binding is calculated by determining nonspecific binding with 25 μM tested plant essential oils and subtracting nonspecific binding from total binding, and the relative binding is plotted using the specific binding from cells treated with tested plant essential oils and solvent (ethanol) treatment as 100;

FIG. 9 is a bar graph depicting the stimulation and inhibition of cAMP levels in cells stably expressing TyrR in response to treatment with tested plant essential oils; wherein S2 cells stably expressing TyrR are treated with 300 μM IBMX and the effect of tested plant essential oil (25 μM) on basal cAMP levels is measured; and

FIG. 10 is a series of graphs depicting the effect of tested plant essential oils on intracellular calcium [Ca²⁺]ᵢ levels in S2 cells stably expressing pAC-TyrR; wherein S2 cells transfected with the empty plasmid are used in parallel for comparison, cells are incubated for 30 s before the addition of 25 μM plant essential oil, the arrow indicates addition of tested agent, and the fluorescence ratio determined from excitation with 340 nm and 380 nm is plotted to indicate sustained increase in [Ca²⁺]ᵢ levels.

### DETAILED DESCRIPTION OF THE INVENTION

The present invention includes compositions, methods, cell lines and reports related to controlling insects via the tyramine receptor and other receptors of the invertebrate olfactory cascade, most preferably the insect olfactory cascade.

The present invention includes a method for screening compositions for insect control activity, reports containing compositions identified by the screening method, and compositions identified by the screening method.

The present invention includes a Drosophila Schneider 2 cell line stably transfected with tyramine receptor that is amplified from Drosophila melanogaster head cDNA phage library (pAc5.1/V5-His B-tyramine receptor).

The present invention includes compositions for controlling insects and methods for using these compositions to control insects. The compositions may include one or more plant essential oils or monoterpenoids of plant essential oils. The compositions may include: thymol, carvacrol, isopar M, mineral oil, methyl salicylate, benzyl alcohol, linalool, arbanol, thyme oil, lilac flower oil, black seed oil and/or an oil named in co-assigned U.S. patent application Ser. No. 10/832,022, now U.S. Pat. No. 7,541,155, which is incorporated herein by this reference. The compositions may include compounds having a monocyclic, carbocyclic ring structure having six-members and substituted by at least one oxygenated or hydroxyl functional moiety. The compositions may include compounds having a six member carbon ring and having substituted thereon at least one oxygenated functional group. The compositions may include compounds having a six member carbon ring and having substituted thereon a hydroxyl group. The compositions may include compounds having a six member carbon ring and having substituted thereon a hydroxyl group on the position 2 or 3 of the ring.

The compositions may include a mixture comprising: about 50% Isopar M, about 20% mineral oil; and about 30% methyl salicylate. The compositions may include a mixture comprising: about: 30% methyl salicylate; about 50% benzyl alcohol; and about 20% linalool. The compositions may include a mixture comprising: about: 70% benzyl alcohol and about 30% arbanol. Said mixtures, combined relative to one another in the percentages set forth, may be combined with additional compounds, as desired. For example, the compositions may include one or more of said mixtures combined with another mixture containing: about 40% thyme oil and about 70% lilac flower oil; or about 50% black seed oil and about 50% lilac flower oil. The compositions including mixtures of compounds may produce synergistic effects, as compared to compositions including fewer of the compounds.

The present invention includes methods for controlling insects by providing compositions that repel or are toxic to insects, arachnids, larvae, and like invertebrates, including: crawling insects, such as American cockroaches and carpenter ants, and drosophila melanogaster fly.

The present invention includes methods for controlling insects by targeting the tyramine receptor of the insect, inducing subsequent cellular changes down stream to the receptor. The subsequent cellular changes may include altered intracellular cAMP levels, Ca²⁺ levels or both.

In some embodiments of the invention, the screening method for insect control activity can target an insect olfactory receptor protein. The insect olfactory system includes more than 60 identified olfactory receptors. These receptors are generally members of a large family of G protein coupled receptors (GPCRs).

In Drosophila melanogaster, the olfactory receptors are located in two pairs of appendages located on the head of the fly. The family of Drosophila chemoreceptors includes approximately 62 odorant receptor (Or) and 68 gustatory receptor (Gr) proteins, encoded by families of approximately 60 Or and 60 Gr genes through alternative splicing. Some of these receptor proteins have been functionally characterized, while others have been identified by sequence homology to other sequences but have not been fully characterized. Other insects have similar olfactory receptor proteins.

In certain embodiments, the insect olfactory receptor protein targeted by the screening or insect control method of the invention is the tyramine receptor (tyrR). In additional embodiments, the insect olfactory receptor protein is the insect olfactory receptor protein Or83b or Or43a. In additional embodiments, the targeted protein can be any of the insect olfactory protein receptors.

Additionally, other components of the insect olfactory receptor cascade can be targeted using the method of the invention in order to identify useful insect control compounds. Exemplary insect olfactory cascade components that can be targeted by methods of the invention include but are not limited to serotonin receptor, Or22a, Or22b, Gr5a, Gr21a, Gr61a, β-arrestin receptor, GRK2 receptor, and tyramine β-hydroxylase receptor, and the like.

The methods of embodiments of the invention can used to control any type of insect. Exemplary insects that can be controlled include but are not limited to beetles, cockroaches, flies, ants, insect larvae, bees, lice, fleas, mosquitoes, moths, and the like. Exemplary insect orders can include but are not limited to Anoplura, Orthoptera, Hemiptera, Ephemeroptera, Strepsiptera, Diptera, Dermaptera, Diplura, Dictyoptera, Collembola, Coleoptera, Neuroptera, Thysanoptera, Mecoptera, Lepidoptera, Ephemeroptera, Plecoptera, Embioptera, Trichoptera, Hymenoptera, Psocoptera, Phasmida, Protura, Thysanura, Mecoptera, Isoptera, Siphonaptera, Mallophaga, Lepidoptera, and the like.

Any insect cell or cell line can be used for the screening assay. Exemplary insect cell lines include but are not limited to SF9, SF21, T.ni, Drosophila S2 cells, and the like. Methods of culturing the insect cells are known in the art, and are described, for example, in Lynn et al., J. Insect Sci. 2002; 2: 9, incorporated herein by reference in its entirety. Methods of starting a new insect cell culture from a desired insect cell are described, for example, in Lynn et al. Cytotechnology. 1996; 20:3-1 1, which is incorporated herein by reference in its entirety.

The present invention is further illustrated by the following specific but non-limiting examples. The following examples are prophetic, notwithstanding the numerical values, results and/or data referred to and contained in the examples.

### EXAMPLES

### Insects and Test Agents

Drosophila melanogaster (wild type) is purchased from Carolina Biological Supply Company (Burlington, N.C.). The tyramine receptor mutant (TyrR^{neo30}) Drosophila melanogaster is obtained from Bloomington Drosophila Stock Center (stock # BL-10268). The mutant flies are constructed in which the insertion of a single P transposable element has caused a mutation in tyramine receptor; their phenotype includes olfaction defects. See e.g., Cooley, et al., (1988) Science, 239, 1121-1128. The responsible transposon is reported as P {hsneo}TyrR^{neo30}, BDGP:1(3)neo30 as described on the flybase website (http://flybase.bio.indiana.edu/.bin/fbidg.htm?FBa10011043). Both Drosophila strains are maintained under standard laboratory conditions.

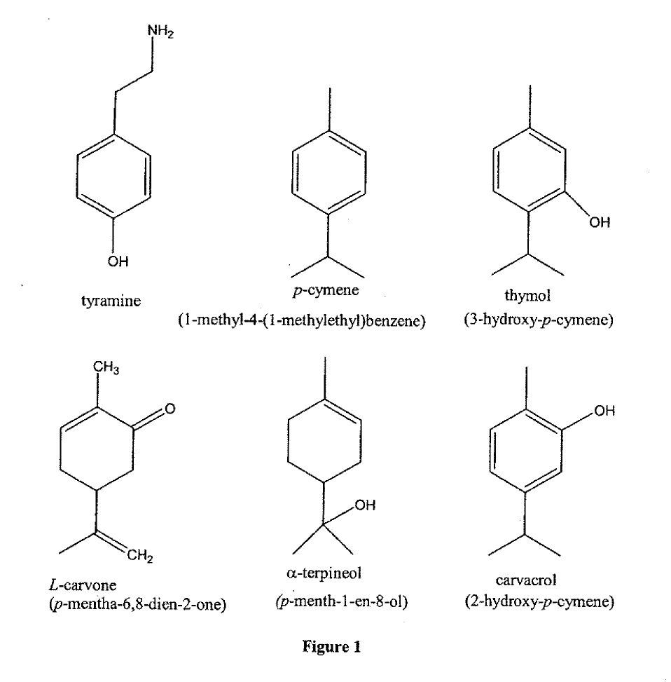

Plant essential oils, such as those depicted in FIG. 1, including: p-cymene (1-methyl-4-(1-methylethyl)benzene), 3-hydroxy p-cymene (thymol), 2-hydroxy p-cymene (carvacrol), p-menth-1-en-8-ol (a-terpineol), and p-mentha-6,8-dien-2-one (L-carvone) are purchased from City Chemical (West Haven, Conn.).

### PCR Amplification and Subcloning of Drosophila melanogaster Tyramine Receptor Gene

The tyramine receptor is amplified from Drosophila melanogaster head cDNA phage library that is obtained through the Berkeley Drosophila Genome Project (www.fruitfly.org). Phage DNA is purified from this library using a liquid culture lysate as described in Lech (2001) “Preparing DNA from small-seal liquid lysates” Ausubel, J. G., Smith, J. A., Struhl, K. (Eds.), Current Protocols in Molecular Biology. John Wiley & Sons, Inc, pp. 1.13.7. Briefly, gene specific primers used to amplify the open reading frame of the Drosophila tyramine receptor (TyrR) are designed based on the published dro-tyr sequence by Saudou et al., (1990, Genbank accession # X54794; protein accession # “CAA38565”). These gene specific primers consist of the 5′ oligonucleotide: 5′ gccgaattcATGCCATCGGCAGATCAGATCCTG3′ (SEQ ID NO. 1) and 3′ oligonucleotide: 5′ taatctagaTCAATTCAGGCCCAGAAGTCGCTTG 3′ (SEQ ID NO. 2). Capitalized letters match the tyramine receptor sequence. The 5′ oligonucleotide also contains an EcoR I site and the 3′ oligonucleotide a Xba I site restriction sites that are indicated by underlined nucleotides. PCR is performed using Vent polymerase (New England Biolabs) with the following conditions: 95° C., 5 min for 1 cycle; 95° C., 30 s; and 70° C., 90 s for 40 cycles; and 70° C., 10 min for 1 cycle. The PCR product is digested with EcoR I and Xba I, subcloned into pcDNA3 and sequenced on both strands by automated DNA sequencing (Vanderbilt Cancer Center). For expression in Drosophila Schneider S2 cells, the tyramine receptor (TyrR) open reading frame is excised from pcDNA3 and inserted into pAc5.1/V5-His B (pAC) using the EcoR I and Xba I restriction sites.

### Cell Culture and Transfection

Drosophila Schneider 2 (S2) cells, lacking endogenous tyramine receptor (Vanden Broeck et al., 1995; Van Poyer et al., 2001), are used in the current study for stable transfection and expression of tyramine receptor that is amplified from Drosophila melanogaster head cDNA phage library. In this regard, cells are grown in Schneider's Drosophila Medium containing 10% heated-inactivated fetal bovine serum (FBS). Medium is supplemented with 50 Units penicillin G/ml, 50 μg streptomycin sulfate/ml. For stable transfection, Drosophila S2 cells are transfected with pAc5.1/V5-His B-tyramine receptor (pAC-TyrR) using the calcium phosphate-DNA coprecipitation protocol as described by Invitrogen Drosophila Expression System (DES) manual. The cells are maintained and grown at room temperature (23° C.) in the same medium supplemented with selection agent (25 μg blasticidin/ml medium). Ten clones of stably transfected cells are selected and separately propagated. Clonal cell lines are selected and assayed for receptor expression with whole cell binding by incubating 1×10⁶ cells in 1 ml insect saline buffer (170 mM NaCl, 6 mM KCl, 2 mM NaHCO₃, 17 mM glucose, 6 mM NaH₂PO₄, 2 mM CaCl₂, and 4 mM MgCl₂) with 4 nM ³H-tyramine for 20 min at 23° C.

Cells are pelleted by centrifugation, washed once with insect saline, and then transferred to scintillation vials. Nonspecific binding is determined by including 100 μM unlabeled-tyramine in the reaction. ³H-tyramine demonstrated the highest binding affinity to Clone #2 cells stably transfected with pAC-TyrR. This clonal cell line, therefore, is propagated and used throughout the study. In all studies, S2 cells transfected with an empty vector lacking the insert are used in parallel as negative controls.

### Membrane Preparation and Radioligand Binding Assay

All steps are performed at 4° C. or on ice. Cells grown in Drosophila media are harvested in the same media by scraping from the dishes and then rinsing dishes with PBS. The cells are centrifuged at 1000 g for 3 min, washed once with PBS and centrifuged again as previously described. The cells are suspended in ice cold hypotonic buffer (10 mM Tris-Cl, pH 7.4), incubated on ice for 10 min, and lysed using a glass dounce homogenizer and tight glass pestle (Kontes Glass Co., Vineland, N.J.) with 10 strokes. Nuclei are pelleted by centrifugation at 600 g for 5 min. The supernatant is decanted and centrifuged at 30,000 g for 30 min to pellet a crude membrane fraction. The pellet is suspended in binding buffer (50 mM Tris-Cl, 5 mM MgCl₂, pH 7.4). Protein concentration is determined by the Bradford Assay (Bio-Rad Laboratories, Hercules, Calif.). Membranes are frozen on dry ice then stored at −75° C. in aliquots.

For the receptor binding assay, radioligand binding is performed in 500 μl binding buffer containing 10-50 μg membrane protein and 4 nM ³H-tyramine. The binding reaction is incubated at 23° C. in the presence and absence of 10 μM unlabeled tyramine. Reactions are terminated after 60 min by addition of 3 ml ice cold binding buffer and filtered over GF/C filters that has been soaked for 30 min in 0.3% polyethylenimine. Filters are rinsed one additional time with 3 ml binding buffer. For the determination of K_{d} and Bₘₐₓ, a range of ³H-tyramine is used from 0.1 to 30 nM, and 10 μM unlabeled tyramine is used as a competitor to determine nonspecific binding. To determine Kᵢ of different ligands, 4 nM ³H-tyramine is used with a concentration range of competitor that gave from 0 to 100% competition. The binding activity of tyramine receptor is also studied in the presence and absence of five plant essential oils, shown in FIG. 1. Binding data is analyzed by Scatchard plots using the software GraphPad Prism (San Diego, Calif.). All binding analyses are performed 3 times with duplicate samples in each assay.

### cAMP Assay

Twenty four hours before cell treatment, 300,000 cells from clone #2 and control cells are plated in 1 ml media with 25 μg blasticidin/ml into 12 well dishes (4.5 cm²). For cell treatment, the media is aspirated and 1 ml PBS with 300 μM IBMX (3-isobutyl-1-methylxanthine) and the test reagent is added. Cells are incubated at 23° C. in the presence and absence of different concentrations of TA. After 10 min incubation, the PBS is aspirated and cells are incubated with 70% ethanol for 12 hours at −20° C. The cellular debris is centrifuged, and then the supernatant is removed and lyophilized to dryness. The amount of cAMP in the extract is determined by using a cAMP binding protein from the ³H-cAMP Biotrak Assay System (Amersham Biosciences, Piscataway, N.J.) as per the manufacturer's instructions. Cell treatment is performed 3 times with duplicates at each concentration. To test the effects of calcium chelation on cAMP levels, the cells are incubated with 20 μM BAPTA/AM for 10 min before the addition of TA.

### Intracellular Calcium Assay

Cells are washed once with insect saline buffer. Cells are collected by scraping and suspended at 1×10⁶ cells/ml in insect saline buffer with 5 μM Fura-2 AM. Cells are incubated at 23° C. for 1 hr in the dark, centrifuged, suspended in 1 ml insect saline buffer, and used immediately for calcium measurements. A spectrofluoremeter with Felix software from Photon Technology International (Lawrenceville, N.J.) is used for the fluorescence measurements and data collection. This assay is performed four separate times. Data are normalized by dividing each value by the background fluorescence at the beginning of the assay before adding test ligand.

### Toxicity Against Drosophila melanogaster Fly

Quantitative Structure Activity Relationships (QSARs) for five monoterpenoid plant essential oils is determined against wild type and tyramine receptor mutant (TyrR^{neo30}) Drosophila melanogaster strains. Monoterpenoid p-cymene and its isomeric phenolic derivatives, thymol and carvacrol, are used. In addition, monocyclic-unsaturated alcohol (a-terpenoil), and a monocylic di-unsaturated ketone (L-carvone) are also tested. To determine whether the cellular changes in pAC-TyrR cell model (clone # 2) in response to treatment with tested essential oils correlate with their insecticidal activity, a toxicity bioassay is performed against the wild type Drosophila melanogaster fly. Acetonic solutions of plant essential oils are prepared and different concentrations of each, that gave from 10%-100% mortality, are applied by topical application. Three replicates, with 5 flies per replicate, are used for each concentration. Controls treated with the same volume (0.5 μl/fly) of acetone. Data are subjected to probit analysis to determine LD₅₀ value for each chemical as described in Finney (1971) Probit analysis, 3^{rd} edn. Cambridge University Press, London, 333. To determine whether the tyramine receptor is involved in the toxicity of tested plant essential oils, tyramine receptor mutant (TyrR^{neo30}) Drosophila melanogaster strain is topically treated with a dose equivalent to the determined LD₅₀ for wild type strain. The mortality is calculated 24 hrs after treatment. Three replicates and 5 insects per replicate are used for the bioassay of each chemical. This bioassay is repeated five times.

### Amplification of a cDNA Encoding a Candidate 7 Transmembrane Tyramine Receptor

The cDNA of ˜1.8-kb is isolated and confirmed to encode the tyramine receptor. The open reading frame encodes a protein of 601 amino acids with a predicted molecular mass of ˜64 KDa. Based on alignment comparisons using DNA Star Software Program, both sequences of dro-tyr (Saudou et al., 1990, supra) and the current TyrR are essentially identical, except one residue at location 261 which is cysteine (C) in the dro-tyr sequence (accession # CAA38565) and tyrosine (Y) in the current TyrR sequence. Hydropathy analysis by the method of Kyte and Doolittle, with a window of 9 amino acids, indicates seven potential transmembrane spanning domains. See Kyte and Doolittle, (1982) J. Mol. Biol. 157, 105-132. The BLAST analysis also indicates that the cloned Drosophila melanogaster TyrR is most similar to other biogenic amine receptors.

TyrR is essentially identical to tyr-dro receptor, a tyramine receptor from the fruit fly Drosophila melanogaster (Saudou et al., 1990, supra), and to the same sequence, also designated as oct/tyr receptor (accession P22270) Arakawa, et al., (1990) Neuron 2, 343-354. Protein alignment indicates the cloned TyrR is 66% identical to Amtyrl (Blenau, et al., (2000) J. Neurochem. 74, 900-908), 48% identical to both Tyr-Loc 1 and Tyr-Loc2 (Vanden Broeck, et al., (1995) J. Neurochem, 64, 2387-2395), 49% identical to c. elegans-Tyr2 (Rex, et al., (2002) J. Neurochem. 82, 1352-1359), 50% identical to Tyr-Bombyx mori (Ohta, et al., (2003) Insect Mol. Biol. 12(3), 217-223), 56% identical to Tyramine receptor from Anopheles gambiae (Genbank, 2003, accession number EAA07468), 29% identical to locus OAR2 (Gerhardt, et al., (1997) Mol. Pharmacol. 51, 293-300), 27% identical to Pa oa₁, an octopamine receptor from Periplaneta americana (Bischof, et al., (2004) Insect Biochem. Mol. Biol. 34, 511-521), and 32% identical to human a2B adenoreceptor (Lomasney, et al., (1990) Proc. Natl. Acad. Sci. USA 87, 5094-5098).

### Pharmacological Analysis of TyrR

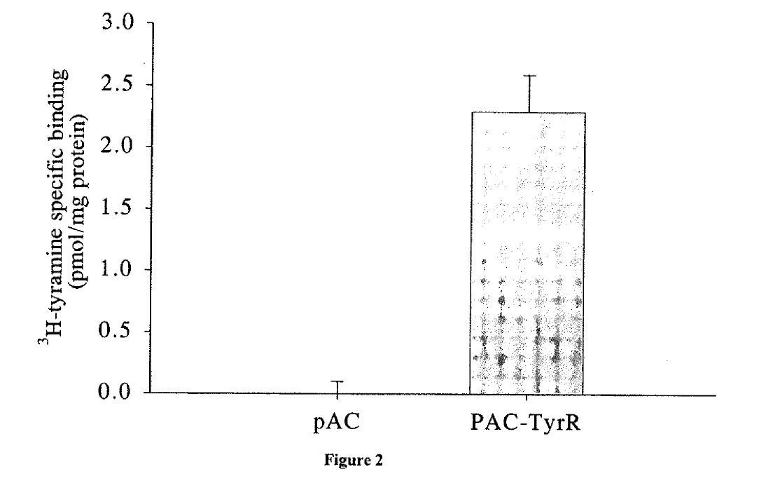

To ensure that selected clonal cells are expressing the TyrR at their surface, the binding of ³H-tyramine to the intact cells is analyzed. Three clones (#2, #3 and #9) demonstrate high binding affinity to ³H-tyramine. ³H-tyramine demonstrates highest affinity to membranes of clone #2 S2 cells transfected with pAC-TyrR, while it does not bind to membranes of S2 cells transfected with an empty vector (pAC) (FIG. 2). Clone #2 cells are propagated and used throughout the study; a stably clonal cell line expressing the D. melanogaster tyramine receptor. The S2 cells transfected with the empty vector are used throughout the study as controls.

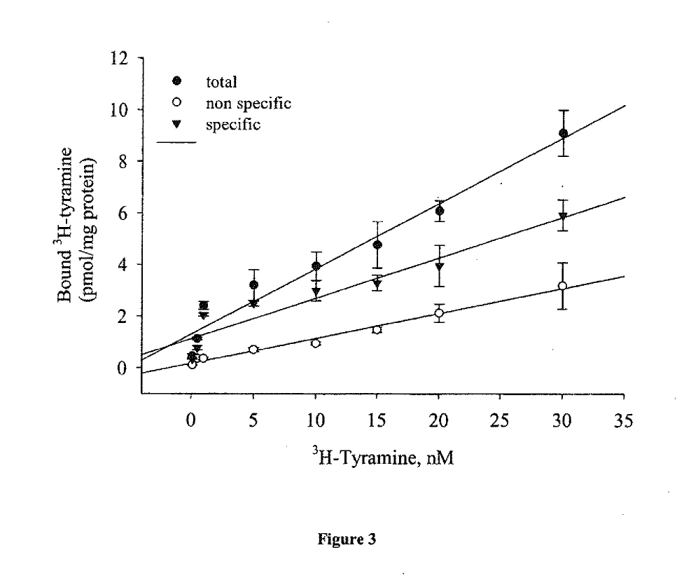

For pharmacological binding experiments, membrane fractions from Drosophila S2 cells expressing TyrR (clone #2) are isolated and analyzed to determine total, nonspecific and specific binding of ³H-tyramine (FIG. 3). The K_{d} and Bₘₐₓ for specific binding are determined to be 2.557 nM and 0.679 pmol receptor/mg membrane protein, respectively. Membrane fractions from Drosophila S2 cells stably transfected with the empty pAC do not demonstrate specific binding. The high affinity binding of ³H-tyramine by the membrane expressing TyrR indicate this is a suitable ligand to be used for competition binding experiments.

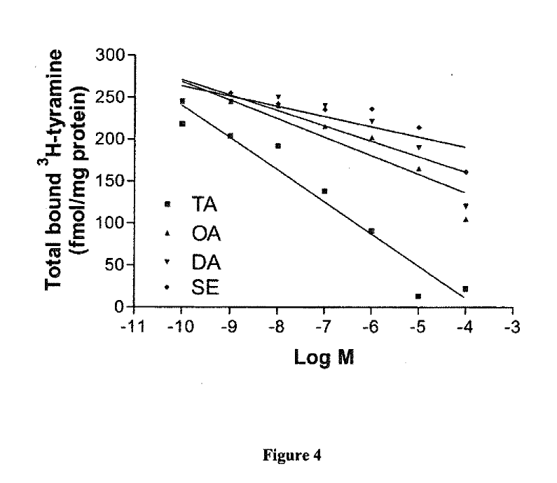

Competitive binding with 5 biogenic amines is utilized to determine the affinities for potential natural ligands of TyrR (FIG. 4 and Table 1). TA has the lowest Kᵢ (1.27 μM) for Drosophila TyrR followed by DL-octopamine (28.68 μM). The decreasing order of affinity for the biogenic amines is TA>OA>dopamine≧serotonin>histamine. These values are about the same as those obtained for tyr-dro (Saudou et al., 1990, supra). In the current study, the affinity of TA is about 22.58 fold higher than DL-octopamine for TyrR. These results therefore indicate that TA is the likely endogenous ligand for the cloned TyrR. The affinity of various biogenic amine receptors antagonists is tested to determine the pharmacological profile of TyrR. The tested drugs demonstrated a potency rank order of decreasing affinity as follows: yohimbine>phentolamine>chlorpromazine>mianserine (Table 1).

TABLE 1

| Chemical Agents | Kᵢ (μM) |
|---|---|
| Biogenic amines | |
| Tyramine (TA) | 1.27 ± 0.08 |
| Octopamine (OA) | 28.68 ± 0.78 |
| Dopamine (DA) | 57.47 ± 3.91 |
| Serotonin (SE) | 89.45 ± 9.01 |
| Histamine (His) | 193.50 ± 16.8 |
| Other ligands | |
| Yohimbine | 0.071 ± 0.001 |
| Phentolamine | 0.125 ± 0.020 |
| Chlorpromazine | 0.193 ± 0.050 |
| Mianserine | 0.280 ± 0.030 |

Inhibition constants of biogenic amines and certain receptor antagonists for competitive binding to TyrR. The inhibition constant (Ki) was determined using membranes from Schneider Drosophila cells that expressed TyrR. The Kis are reported as mean + standard deviation. ANOVA indicated statistically significant differences (p < 0.05) between all pairwise comparisons of biogenic amine Kis as well as other ligands Kis.

This potency order of tested drugs is in agreement with the potency order described by Saudou et al., (1990), supra. Yohimbine is identified as specific antagonist for tyramine receptor from Drosophila melanogaster (Saudou et al., 1990, supra; Arakawa et al., 1990, supra) and Bombyx mori (Khan, et al., (2003) Arch. Insect Biocehm. Physiol. 52, 7-16). The pharmacology of this receptor does not agree with any of the other biogenic amine receptors cloned from Drosophila or in other insect species. In particular, the octopamine receptor cloned from Periplaneta americana, Pa oa₁ or from Drosophila melanogaster, OAMB (Bischof and Enan, 2004, supra), or octopamine receptor characterized in various insect preparation Evans (1981) J. Physiol. 318, 99-122; Dudai, et al., (1982) J. Neurochem. 38, 1551-1558; Guillen, et al., (1989) Life Sci. 45(7), 655-662), did not display an affinity rank order similar to the current data.

### Effects on cAMP Levels by Tyramine Through TyrR

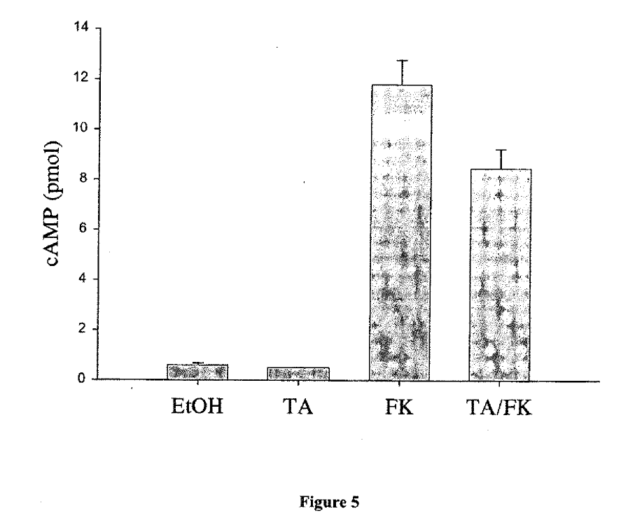

To determine which second messenger signaling pathways TyrR might be coupled to, clone #2 cells or control cells are used. In the control cells that are transfected with plasmid lacking TyrR, TA at concentrations up to 100 μM significant effects on cAMP levels as compared to non-transfected cells are not produced. In clone #2 cells, TA (10 μM) induced 18% decrease in the basal level (0.601±0.080 pmol cAMP) of cAMP (FIG. 5). The data also demonstrate that the TA induced decreases in forskolin-increased cAMP level is dose-dependent with an IC₅₀=5.802 W. Since these assays are performed in the presence of the phosphodiesterase inhibitor IBMX, the changes in cAMP levels are concluded to be through changes of adenylate cyclase activity.

### Effects on Intracellular Calcium [Ca²⁺]ᵢ Levels by Tramine Through TyrR

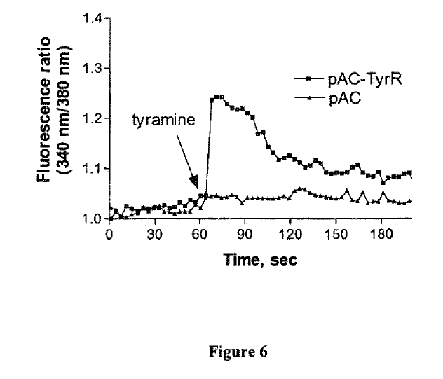

The ability of TA to modulate calcium levels in clone #2 cells is determined. A rapid increase in [Ca²⁺]ᵢ is detected when 1 μM TA is added to these cells (FIG. 6). Testing of this amine at additional concentrations indicates that the lowest concentration of TA that can increase [Ca²⁺]ᵢ levels is 20 nM. On the other hand, in S2 cells transfected with pAC lacking an insert, 10 μM TA does not increase the level of [Ca²⁺]ᵢ beyond its effect in untransfected S2 cells. This result is similar to that obtained with the cAMP assay in that S2 cells transfected with pAC lacking an insert did not respond to TA at 100 μM. This data is consistent with clone #2 cells expressing a functioning tyramine receptor.

### Receptor Coupling to Second Messenger Systems

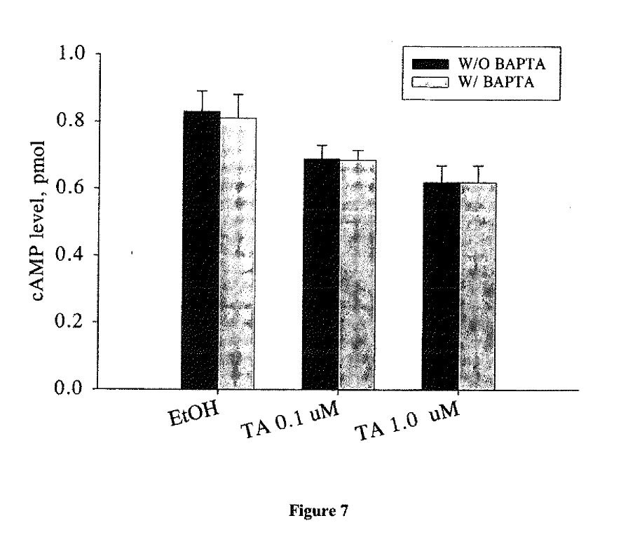

The current data raises the possibility that the coupling of the cloned Drosophila-tyramine receptor to the multiple signaling systems is direct, or that the changes in cAMP levels could be secondary to changes in [Ca²⁺]ᵢ levels. Therefore, to rule out the possibility that receptor activation triggers increase in intracellular calcium level, which then initiates a secondary change in cAMP levels, an experiment is performed in the presence of an intracellular calcium chelator BAPTA/AM. BAPTA/AM at 20 μM is found to inhibit the increase in free [Ca²⁺]ᵢ when 1 μM TA is added to the TyrR expressing cells. Therefore, TA mediated changes in cAMP levels are compared in the absence and presence of 20 μM BAPTA/AM. The levels of cAMP following treatment with either 100 nM or 1 μM TA as well as basal cAMP levels are not found to be statistically different whether in the absence or presence of 20 μM BAPTA/AM (FIG. 7) (P>0.05 for all 3 treatments). Therefore, the decrease in cAMP level by TA likely results from direct coupling of TyrR to a G-protein that leads to down-regulation of adenylate cyclase.

### Correlation Between Cellular Chances and Insecticidal Activity in Response to Treatment with Plant Essential Oils

The present study is designed to address the impact of availability and location of hydroxyl group within the chemical structure of selected plant essential oils on the tyramine receptor signaling cascade. In addition, this study is designed to determine whether the chemicals-receptor interaction correlates with their insecticidal activity.

### Effect on Receptor Binding Activity

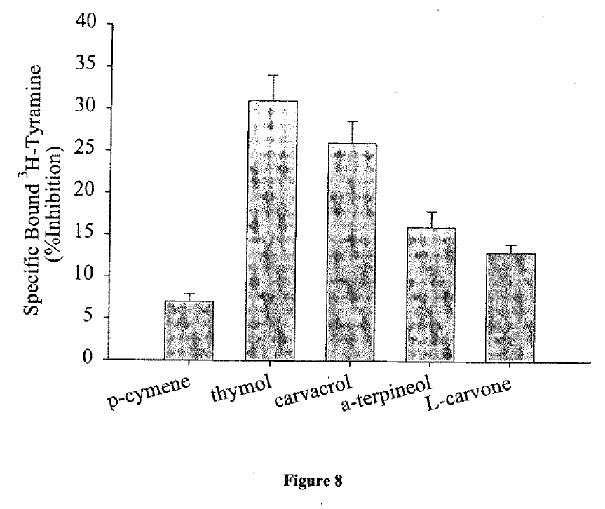

The binding activity of ³H-tyramine to membranes expressing the tyramine receptor is performed in the presence and absence of five plant essential oils. These chemicals are selected based on their insecticidal activity and the absence or presence and location of the hydroxyl group within the molecule. Membrane protein (10 μg) is incubated with 4 nM ³H-tyramine in the presence and absence of 25 μM of the test chemical. The specific activity is calculated as the difference between counts in the presence and absence of test chemical. P-cymene, which lack the hydroxyl group, induces slight inhibition (7%) in tyramine receptor binding activity as compared to the control value (1.614±0.22 pmol/mg protein). A significant (P<0.05) decrease in receptor binding activity in response to treatment with thymol and carvacrol is found (FIG. 8). While thymol (3-hydroxy-p-cymene) induces 31% inhibition in the receptor binding activity, 26%, 16%, and 13% decrease in receptor binding activity is found after treatment with carvacrol (2-hydroxy-p-cymene), a-terpineol (p-menth-1-en-8-ol) and L-carvone (5-methyl-5-(1-methylethylene)-2-cyclohexane-1-one), respectively.

### Effect on cAMP Production

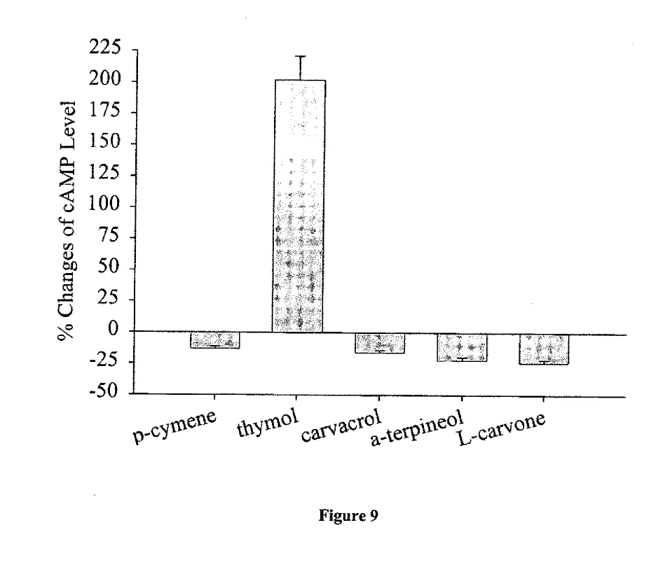

Tested plant essential oils induces changes in the cAMP level (FIG. 9). Thymol increases (by 202%) the cAMP production in cells expressing the tyramine receptor as compared to the cAMP levels (0.9056±0.078 pmol cAMP) in cells treated with the solvent (ethanol) only. A significant (P<0.05) decrease in cAMP production is found in response to treatment with carvacrol (16%), L-carvone (24%) and a-terpineol (22%). The decrease in cAMP in response to treatment with p-cymene (13%) is not significant. TA (FIG. 5) induces 18% decrease in cAMP level in clonal cells expressing tyramine receptor.

### Effect on Intracellular Calcium [Ca²⁺]ᵢ Mobilization

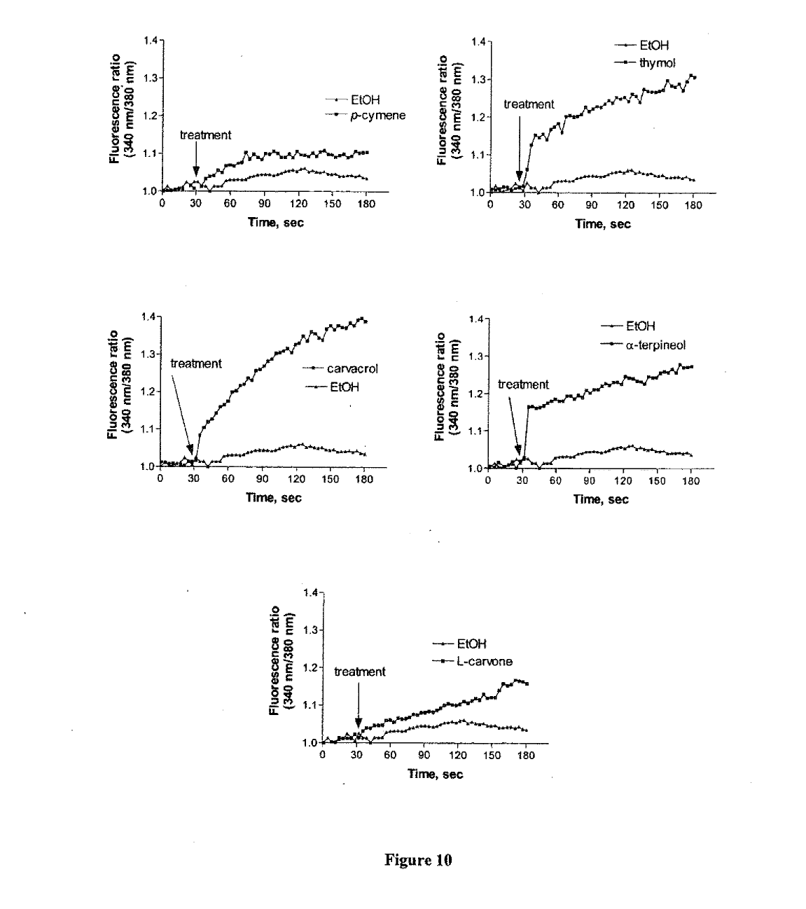

To address whether changes in [Ca²⁺]ᵢ in the TyrR-expressing transfected cells in response to tested plant essential oils is mediated specifically through the TyrR, the TyrR clonal cells are treated with the same volume of ethanol (solvent, 1 μl) and changes in [Ca²⁺]ᵢ is monitored. As demonstrated in FIG. 10, the changes in [Ca²⁺]ᵢ in response to EtOH in these cells are about the same as the changes in [Ca²⁺]ᵢ in cells transfected with the empty plasmid (pAC, FIG. 6). On the other hand, a remarkable increase in [Ca²⁺]ᵢ is found in response to treatment with carvacrol, thymol and a-terpineol as compared to treatment with ETOH (FIG. 10). Carvacrol is the most potent chemical that induces increase in [Ca²⁺]ᵢ, followed by thymol, a-terpineol, L-carvone, then p-cymene. Both thymol and carvacrol induce more increases in [Ca²⁺]ᵢ than TA. The increase in [Ca²⁺]ᵢ in response to a-terpineol is similar to that induced by TA.

The data suggests that elevation pattern of [Ca²⁺]ᵢ levels is chemical-dependent. While application of TA induces an immediate but transient peak (˜20 s) in [Ca²⁺]ᵢ level (FIG. 6), the peaked [Ca²⁺]ᵢ level is slower in onset and has a longer recovery time (more than 3 min) in response to treatment with tested plant essential oils. In addition, the increase in [Ca²⁺]ᵢ level in response to p-cymene and L-carvone is slower than thymol, carvacrol and a-terpineol (FIG. 10). Thus, the efficacy of coupling of this cloned tyramine receptor to different second messenger signaling varies with the chemical used.

### Toxicity Against Wild Type and Tyramine Receptor Mutant (TyrR^{neo30}) Drosophila melanogaster Fly

To determine whether the tyramine receptor mediates the toxicity of tested plant essential oils, Drosophila wild type strain vs. tyramine receptor mutant (TyrR^{neo30}) strain are used in the bioassay test. Based on the calculated LD₅₀ values (Table 2), thymol is the most toxic chemical (LD₅₀=0.9 μg/fly) against wild type Drosophila melanogaster strain, followed by carvacrol (LD₅₀=1.4 μg/fly), L-carvone (LD₅₀=2.3 μg/fly), a-terpineol (LD₅₀=12.0 μg/fly), p-cymene (LD₅₀=15.5 μg/fly). The toxicity of thymol and carvacrol is abolished when they are topically applied to the TyrR^{neo30} strain (Table 2) which suggests that insertion of the P element completely abolishes the response of the tyramine receptor to both chemicals.

TABLE 2
Insecticidal activity of certain plant essential oils against tyramine
receptor mutant (TyrR^{neo30}) Drosophila melanogaster

| | Wild type- | Mortality at LD₅₀ of | |
|---|---|---|---|
| | calculated | wild type Drosophila | |
| | LD₅₀ values | melanogaster strain | |
| Chemical Name | μg/fly) | Wild type | TyR^{neo30} |
| p-cymene | 15.5 | 57.3% | 56.0% |
| 3-hydroxyl-p-cymene (thymol) | 0.9 | 62.7% | 0.0% |
| 2-hydroxy-p-cymene | 1.4 | 69.3% | 0.0% |
| (carvacrol) | | | |
| trimethyl-3-cyclohexene-1- | 12.0 | 46.7% | 26.7% |
| methanol (a-terpineol) | | | |
| p-mentha-6,8-diene-2-one (L- | 2.3 | 37.3% | 38.7% |
| carvone) | | | |

The LD₅₀ values of tested chemicals against wild type Drosophila were topically applied against wild type and tyramine receptor mutant (TyrR^{neo30}) strains. Mortality was determined 24 h after treatment. Data are the average of three replicates, 5 flies per replicate. This experiment repeated five times.

The toxicity of a-terpineol is decreased against TyrR^{neo30} strain. However, mutation of the tyramine receptor does not affect the toxicity of L-carvone and p-cymene (Table 2). Collectively, the toxicity data is consistent with the cellular changes induced in clonal cell line expressing the tyramine receptor in response to treatment with tested plant essential oils. In conclusion, the data suggest a correlation between agent-induced cellular changes in S2 cells expressing TyrR and their insecticidal activity.

### Validation of the Cell Model Expressing the TA Receptor

Biogenic amines play a vital role in the survival and behavior of invertebrates, thus, one aspect of the present invention is a cell model that can be used to study the molecular interaction of plant essential oils with biogenic amine receptors. In this regard, the tyramine receptor was isolated and amplified from Drosophila melanogaster head cDNA phage library. A permanent clonal cell line was then developed using Drosophila Schneider 2 (S2) cells. These cells are often used as host cells for stable expression and functional characterization of mammalian and insect G-protein coupled receptors (GPCRs) such as biogenic amine receptors. Van Poyer, et al., (2001) Insect Biochem. Mol. Biol. 31, 333-338 demonstrated the presence of an endogenous mRNA encoding an octopamine receptor type in S2 cells, but there was no evidence to demonstrate the presence of the tyramine receptor in these cells.

No specific receptor binding activity to ³H-tyramine is found in membranes isolated from S2 cells transfected with pAC lacking the insert (TyrR). Also, no significant changes in either the [Ca²⁺]ᵢ level or cAMP level in these cells are found in response to TA treatment as compared to changes in untransfected S2 cells. On the other hand, using the TyrR cloned S2 cell model, TA is found to inhibit ³H-tyramine binding, and result in decreased cAMP levels and increased [Ca²⁺]ᵢ. Since the increased [Ca²⁺]ᵢ is detected immediately upon TA addition, this raises the question of whether the increased [Ca²⁺]ᵢ is leading to a secondary decrease in cAMP levels. The finding that demonstrates BAPTA/AM does not affect the forskolin-induced changes in cAMP levels suggests that the coupling of the cloned TyrR from Drosophila head to the changes in cAMP levels is direct, and not a secondary consequence of changes in [Ca²⁺]ᵢ levels.

The possibility that single agonist can interact with different subtypes of GPCRs, each of which can be coupled to a specific second-messenger system, is not likely. This conclusion is based on the TA binding studies with cell membranes that express the tyramine receptor. The study using polymerase chain reaction (PCR) analysis of transfected S2 cell mRNA and closely spaced overlapping primer pairs gives no evidence of the production of multiple transcripts. In addition, the findings described in these examples are in agreement with those from an earlier reports of the cloned Drosophila octopamine/tyramine receptor in different cell lines (Saudou et al., 1990, supra; Robb, et al., (1994) Embo. J. 13, 1325-1330).

Furthermore, Evans et al., (1995) Progress Brain Res. 106, 259-268 reported that the TA-induced inhibition of forskolin-increased cAMP levels in cells expressing Drosophila octopamine/tyramine receptor, is mediated by a pertussis toxin-sensitive G-protein coupled receptor pathway, while its elevation in [Ca²⁺]ᵢ levels is mediated via an independent pathway which is pertussis toxin-insensitive.

### Downstream Effects of Treatment with Plant Essential Oil Monoterpenoids in Clonal Cell Model

Following the validation of the clonal cell model expressing the tyramine receptor, the interaction of selected plant essential oil monoterpenoids with the tyramine receptor and the subsequent cellular changes down stream to the receptor in these cells are studied. The study also addresses the structural features of the chemical entity required in compound-receptor interaction.

Grodnitzky and Coats, (2002) Am. Chem. Soc. Chapter 23, 238-250 found that among several classical and quantum parameters chosen to represent the features of molecules that are important in receptor-ligand interaction, only a correlation between two parameters were found: electronic properties (Mulliken population) within each monoterpenoid compound, and their toxicity against housefly. Their data suggested that the size or shape of monoterpenoid chemicals is not a major factor on toxicity against housefly.

In addition, Aoyama et al., (2001) Arch. Insect Biochem. Physiol. 47, 1-7 studied the impact of substitution of octopamine and tyramine derivatives on cAMP production in Bombyx mori (silkworm). The data indicated that none of the derivatives tested increased cAMP level more than the parent compounds. However, the octopamine derivative that bears a chlorine atom at p-position and a hydroxyl group at β-position was among the most effective analogous. In addition, substitution of p-hydroxyl group on TA with a chlorine atom decreased the efficiency of the derivative on cAMP production.

In the studies described in these examples, five structurally related plant essential oil monoterpenoids are selected, three of them (p-cymene, thymol and carvacrol) are differing by the position of only a single hydroxyl group on the ring, a mono-unsaturated alcohol (a-terpineol) and di-unsaturated ketone (L-carvone). In cells expressing the tyramine receptor, their interaction with the receptor and subsequent cellular changes down-stream to the receptor are determined. Their toxicity against wild type and mutant TyrR^{neo30} Drosophila strains is also determined. The data demonstrates that the presence and location of the hydroxyl group on the ring induces specific conformational changes in the receptor, which suggest that it may couple preferentially to separate G-protein.

P-cymene, for example, which lacks the hydroxyl group, induces a slight decrease in ³H-tyramine-receptor-binding, an insignificant decrease in cAMP level, and a slight increase in [Ca²⁺]ᵢ level. Its toxicity against wild type Drosophila melanogaster is low as compared to other tested chemicals.

Thymol, on the other hand, induces a significant decrease in receptor binding activity, and a significant increase in cAMP level (202%), and a remarkable increase in [Ca²⁺]ᵢ level.

Carvacrol decreases the receptor binding activity, significantly decreases cAMP level, and markedly increases [Ca²⁺]ᵢ level in cells expressing the tyramine receptor. Both chemicals demonstrate high toxicity against wild type Drosophila melanogaster. Their toxicity is abolished against Drosophila TyrR^{neo30} strain.

The opposite changes in cAMP level in response to treatment with thymol and carvacrol reflects the complexity in the coupling of the cloned tyramine receptor to the two second messenger pathways. This is demonstrated in the present study in which both chemicals that differ structurally by only the location of a hydroxyl group on the ring can differentially couple the receptor to two second messenger systems. Since the cloned Drosophila tyramine receptor can be differentially activated by chemicals differing by only a single hydroxyl group, it has the advantage that site-directed mutagenesis can be used to identify the key amino acid which interacts with the hydroxyl group.

This single interaction is likely to be responsible for the initiation of the conformational change in the receptor that leads to the switching of its coupling to the second messenger cascades. The present studies are therefore consistent with the early findings that the cellular response of GPCRs strictly relies on the specificity of interaction between the receptor and the G-protein. See Gudermann, et al., (1996) Annu. Rev. Pharmacol. Toxicol. 36, 429-459; Blenau, et al., (2001) Arch. Insect Biochem. Physiol. 48, 13-38.

Following treatment with thymol, the increase in cAMP level in cells expressing the tyramine receptor can be attributed to the binding of the receptor to Gs-type protein. The activated Gas subunit will interact with adenylyl cyclase in the plasma membrane resulting in an increase of adenylyl cyclase activity and production of cAMP from ATP.

On the other hand, the decrease in cAMP level by carvacrol is mediated by interaction of the receptor with inhibitory G-proteins (Gi). Interaction of adenylyl cyclase with activated Gai subunits most likely competes with binding of activated Gas subunits and thereby interferes with cyclase activation. The importance of the hydroxyl group on the ring is also supported by the finding that in cells expressing tyramine receptor, p-cymene (lacks hydroxyl group) do not induce a significant change in the signaling cascades down-stream to the receptor.

Other evidence reflecting the impact of the presence and location of the hydroxyl group on the toxicity emerge from cellular changes in cells expressing TyrR and toxicity of a-terpineol and L-carvone. a-terpineol, induces a 16% decrease in receptor binding activity, a 22% decrease in [cAMP]ᵢ levels and a remarkable increase in [Ca²⁺]ᵢ. L-carvone, induces an insignificant decrease in tyramine receptor binding activity, a significant decrease in [cAMP]ᵢ level, and a remarkable increase in [Ca²⁺]ᵢ level. The toxicity of L-carvone against wild type Drosophila melanogaster strain is about 5-fold more than a-terpineol. While the toxicity of a-terpineol against the TyrR^{neo30} strain is decreased, this mutation does not affect the toxicity of L-carvone. These data suggest that both a-terpineol and L-carvone activate the second messenger pathways through another biogenic amine receptor such as the octopamine receptor.

In conclusion, the present studies show that the tyramine receptor mediates the insecticidal properties of thymol and carvacrol and, in part, the toxicity of a-terpineol against Drosophila melanogaster fly.

The present studies show that an electronegative group, such as hydroxyl group, on the position 2 or 3 of the ring of plant essential oils is beneficial for their insecticidal activity through tyramine receptor. The structural features required for the accommodation of functional groups of the molecule by the receptor binding sites may be characterized. The present studies provide potential biomarkers that can be used to study the correlation between biochemical and molecular changes in cells expressing TyrR as well as the insecticidal activity of test agents.

These studies add insight into the molecular mechanisms of action of plant essential oils, may lead to an understanding of the signaling pathways involved in the regulation of insect's multiple physiological functions such as muscular systems, sensory organs, and endocrine tissues, as well as learning and behavior, which be used to aid in identification of novel targets for the development of molecularly targeted and environmentally safer pesticides.

The foregoing specific but non-limiting examples are included herein to illustrate the present invention, but are prophetic, notwithstanding the numerical values, results and/or data referred to and contained therein.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification and Example be considered as exemplary only, and not intended to limit the scope and spirit of the invention.

Unless otherwise indicated, all numbers expressing quantities of ingredients, properties such as reaction conditions, and so forth used in the Application are to be understood as being modified in all instances by the term “about.” Accordingly, unless indicated to the contrary, the numerical parameters set forth in the Application are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this application, various publications are referenced. All such references are incorporated herein by reference.

What is claimed is:

1. A method of screening compositions for potential invertebrate control activity comprising:
providing a cell expressing a G protein-coupled receptor of the insect olfactory cascade, or fragment thereof, wherein said G protein-coupled receptor is selected from the group consisting of: Or22a, Or22b, Gr5a, Gr21a, and Gr61a,
contacting a test composition to said cell, thereby causing an effect through said receptor,
measuring at least one parameter selected from the group consisting of olfactory cascade receptor binding affinity, intracellular cAMP levels, and intracellular Ca²⁺ levels,
selecting a composition capable of altering at least one parameter selected from the group consisting of: increased olfactory cascade receptor binding affinity, altered intracellular cAMP levels and altered intracellular Ca²⁺ levels; and
classifying the selected composition as having potential invertebrate control activity.

2. The method of claim 1, wherein contacting said composition to said cell increases binding activity of the receptor.

3. The method of claim 1, wherein contacting said composition to said cell alters the level of intracellular cAMP.

4. The method of claim 1, wherein contacting said composition to said cell alters the level of intracellular Ca²⁺.

5. The method of claim 1, wherein said cell is a eukaryotic cell.

6. The method of claim 1, wherein said cell has been transformed with a nucleic acid encoding a tyramine receptor or fragment thereof, with the proviso that the receptor does not comprise a tyramine beta hydroxylase receptor.

7. The method of claim 5, wherein the cell is a Drosophila Schneider (S2) cell.

8. The method of claim 5, wherein the cell is a mammalian cell.

9. The method of claim 8, wherein the cell is selected from the group consisting of a COS-7 cell and an HEK-293 cell.

10. The method of claim 1, wherein said composition comprises a compound derived from a plant.

11. The method of claim 1, wherein said composition comprises a plant essential oil.

12. The method of claim 1, wherein said composition comprises a compound selected from the group consisting of arbanol, t-anethole, black seed oil, camphene, carvacrol, d-carvone, l-carvone, 1,8-cineole, p-cymene, diethyl phthalate, eugenol, geraniol, isopropyl citrate, lemon grass oil, lilac flower oil, lime oil, d-limonene, linalyl anthranilate, linalool, lindenol, methyl citrate, methyl di-hydrojasmonate, myrcene, perillyl alcohol, phenyl acetaldehyde, α-pinene, β-pinene, piperonal, piperonyl, piperonyl acetate, piperonyl alcohol, piperonyl amine, quinone, sabinene, α-terpinene, terpinene 900, α-terpineol, γ-terpineol, 2-tert-butyl-p-quinone, α-thujone, thyme oil, thymol, allyl sulfide, allyl trisulfide, allyl disulfide, anethole, artemisia alcohol acetate, benzyl acetate, benzyl alcohol, bergamotene, β-bisabolene, bisabolene oxide, α-bisabolol, bisabolol oxide, bisabolol oxide β, hornyl acetate, β-bourbonene, α-cadinol, camphene, α-campholene, α-campholene aldehyde, camphor, caryophyllene oxide, chamazulene, cinnamaldehyde, cis-verbenol, citral A, citral B, citronellal, citronellol, citronellyl acetate, citronellyl formate, α-copaene, cornmint oil, β-costol, cryptone, curzerenone, d-carvone, l-carvone, davanone, diallyl tetrasulfide, dihydropyrocurzerenone, β-elemene, γ-elemene, elmol, estragole, 2-ethyl-2-hexen-1-ol, eugenol acetate, α-farnesene, (Z,E)-α-farnesene, E-β-farnesene, fenchone, furanodiene, furanoeudesma-1,3-diene, furanoeudesma-1,4-diene, furano germacra 1,10(15)-diene-6-one, furanosesquiterpene, geraniol, geraniol acetate, germacrene D, germacrene B, α-gurjunene, α-humulene, α-ionone, β-ionone, isoborneol, isofuranogermacrene, iso-menthone, iso-pulegone, jasmone, lilac flower oil, limonene, linalool, linalyl acetate, lindestrene, methyl-allyl-trisulfide, menthol 2-methoxy furanodiene, menthone, menthyl acetate, methyl cinnamate, methyl salicylate, menthyl salicylate, myrtenal, neraldimethyl acetate, nerolidol, nonanone, 1-octanol, E ocimenone, Z ocimenone, 3-octanone, ocimene, octyl acetate, peppermint oil, α-phellandrene, β-phellandrene, piperonal, prenal, pulegone, sabinene, sabinyl acetate, α-santalene, santalol, sativen, δ-selinene, β-sesquphelandrene, spathulenol, tagetone, α-terpinene, 4-terpineol, α-terpinolene, α-terpinyl acetate, α-thujene, thymyl methyl ether, trans-caryophyllene, trans-pinocarveol, trans-verbenol, verbenone, yomogi alcohol, zingiberene, and dihydrotagentone.

13. The method of claim 1, further comprising:
providing a second cell expressing insect receptor olfactory protein Or43a or fragment thereof;
wherein said test composition is also contacted to said second cell.
